(19) United States
(12) Patent Application Publication
(10) Pub. No.: US 2010/0143484 A1
(43) Pub. Date: Jun. 10, 2010

(54) ORAL SUBMICRON PARTICLE DELIVERY SYSTEM FOR PROTEINS AND PROCESS FOR ITS PRODUCTION

(75) Inventors: Ana Catarina Beco Pinto Reis, Coimbra (PT); Franscisco Jose Baptista Veiga, Coimbra (PT); Antonio Jose Rebeiro, Amarante (PT); Ronald James Neufeld, Sydenham (CA); Christiane Damge, Strasbourg (FR)

Correspondence Address: SUGHRUE MION, PLLC, 2100 PENNSYLVANIA AVENUE, N.W., SUITE 800, WASHINGTON, DC, 20037, US

(73) Assignee: UNIVERSIDADE DE COIMBRA, Coimbra (PT)

(21) Appl. No.: 12/447,793

(22) PCT Filed: Oct. 24, 2007
(86) PCT No.: PCT/PT07/00046

§ 371 Date: Jan. 13, 2010

Related U.S. Application Data
(60) Provisional application No. 60/854,086, filed on Oct. 25, 2006.

(51) Int. Cl. A61K 38/28; A61K 9/14; A61K 38/16; A61P 3/10

(52) U.S. Cl. 424/491; 514/12; 514/3

(57) ABSTRACT

The invention provides a novel submicron system for the oral administration of proteins. An effective oral carrier for proteins should shield its content against the gastrointestinal tract proteases and be capable of facilitating the uptake of the protein drug across the gastrointestinal epithelium. The present invention relates to production of gelled particles which comprises a protein drug susceptible to enzymatic degradation by enzymes and acid conditions in the stomach, a polymeric matrix which undergoes precipitation-swelling process, and two-layer-coating materials which are themselves capable of enhancing absorption of said drug across the intestinal mucosal tissues and of inbihiting degradation of said drug by gastric enzymes. Insulin-loaded particles with appropriate submicron size for gastrointestinal absorption were made of natural occurring polymers by emulsification-based method and proved to be gastric pH and protease protective. Effects on glycemia were observed during 14 h after their oral single administration to rats, achieving 42% of pharmacological activity compared to subcutaneous administration. Postprandial rise in blood glucose was suppressed and insulinemia levels increased by a factor of seven. The relative oral bioavailability of insulin calculated over 8 h by comparison with a subcutaneous injection of free insulin was 34%.

### FIELD OF THE INVENTION

This invention relates to the use of polymer-based vehicles useful for delivering therapeutic agents including protein drugs which ordinarily are not easily delivered orally. The field of this invention concerns the development of protein-loaded sub micron particles produced and coated by a novel coating method, where the encapsulation efficiency achieved can be greater than 85%. This oral submicron particle delivery system consists of entrapped protein within a polymer network surrounded by two-layer coating whose characteritics allow to interaction with the stomach or intestinal mucosa to favorably increase the probability of the therapeutic diffusing into the circulatory system. Insulin-loaded submicron particles proved to be gastric pH and protease protective with appropriate size for gastrointestinal absorption. In vivo hypoglycaemic effects in rat model were observed during 14 h after oral single administration, achieving 42% of pharmacological activity compared to subcutaneous administration of insulin solution. Postprandial rise in blood glucose was suppressed and insulinemia levels increased by a factor of seven. The relative oral bioavailability of insulin calculated over 8 h by comparison with a subcutaneous injection of free insulin was 34%. These submicron particles are biodegradable, so that undesired accumulations in the host body are avoided. Moreover, the production method operates primarily at ambient temperatures.

### BACKGROUND OF THE INVENTION

Peptide and protein drugs are attracting increasing interest with better understanding of their role in physiopathology, as well as progress in biotechnology and biochemical synthesis. Many therapeutic proteins have been identified and tested as having significant therapeutic benefits for several diseases such as hormonally based diseases. Unfortunately, many of those disorders are chronic and require continuous administration of the required therapeutic. The use of therapeutic proteins depends on the ability to easily administer them to patients in a controllable and acceptable manner. Obviously, the oral administration would be the most desirable method for administering such materials to patients. However, the oral use of peptides and proteins in medicine has been limited by low bioavailability, which results from their poor stability to proteolytic and hydrolytic degradation, high molecular weight, low permeability across barriers, and short biologic half-life in the circulatory system¹

A typical and very important example of a potential application for an oral protein delivery technology is the treatment of Diabetes Mellitus. Diabetes mellitus is a illness requiring strict glycemic control to reduce its incidence and progression². The development of insulin-dependent diabetes mellitus (IDDM) in human, and in animal models of human disease, is characterized by mononuclear cell infiltration and, beta-cell destruction in the pancreatic islets (insulitis). Insulin replacement therapy provides the most effective and unique means for glycemic control. Replicating physiological insulin secretion as a means of restoring normal metabolism, minimizes complications, and has thus become the essential goal of insulin treatment³.

Currently insulin is daily administered, once or repeated several times, for most patients by parenteral routes. Tritation of doses vary widely from patient to patient, depending on many factors such as body weight, height, the nature of the specific form and severity of diabetes, and the degree of diet control which can be exercised by each patient. It is not possible to identify a single dosage of insulin which can be considered as a standard human dosage form. In addition to the patient acceptability problem associated with delivery of insulin by injection, there are additional difficulties associated such as need for syringes and other medical devices and the excessive amounts of insulin to which various organs or tissues of the body are subjected when insulin is administered by this route. Additionally, several side effects have been associated with parenteral administration of insulin such as lipodystrophy at the site of the injection, lipoatrophy, lipohypertrophy or occasional hypoglycaemia.

As well, in some cases patitents with type II diabetes may require insulin therapy. Diabetes type II is more prevalent than type I and many type II cases are not diagnosed. Those with the disease do not show an absolute deficiency of insulin since their pancreas produce some. Type II diabetes is associated with obesity and aging and it is considered as a lifestyle-dependent disease with a strong genetic component. The insulin action is not normal. Most type II diabetes patients initially have high insulin levels along with high blood sugar. However, since sugar signals the pancreas to release insulin, type II diabetics eventually become resistant to that signal and the endocrine-pancreas soon will not make enough insulin. As the disease progresses the impairment of insulin secretion worsens, and therapeutic replacement of insulin often becomes necessary.

Many of previous problems would be solved by the development of an oral dosage form of insulin which could be administered, and made available in the circulatory system. As a result, after gastrointestinal absorption, insulin should undergo the first-hepatic-bypass, and thus trigger a primary effect by inhibiting hepatic glucose output⁴. However, peroral bioavailability of insulin is relatively low mainly due to high proteolytic activity in the gastrointestinal tract and low permeability of the intestinal epithelium. Several strategies to overcome these barriers for perorally administered insulin include the addition of enzyme inhibitors^{5 6-9} and/or permeation enhancers¹⁰⁻¹⁸, chemical modification^{19-22 23-27}, polymeric carriers^{4, 28-31 26, 32-37}, liposomes³⁸⁻⁴³, emulsions⁴⁴⁻⁴⁷, enteric coating^{14, 48-50}, solid lipid nanoparticles, colon targeting of the drug delivery system^{51, 52} where the enzymatic activity is relatively low⁵³, ileum targeting⁵⁴ or combination of strategies^{55-59 53,60, 61}.

Some of the previous approaches for improving the oral bioavailability of therapeutic proteins and peptides like the permeation enhancers may cause side effects such as systemic toxicity and damage to the epithelium. The potentially invasive nature of this approach combined with the lack of accurate control over the tight junction permeability limits its clinical applicability. As well, enzyme inhibitors may have a toxic potential caused by the inhibition of digestive enzymes, which can further cause incomplete digestion of the nutritive proteins. In addition, this inhibitory action can cause increased secretion of these enzymes by a feed-back mechanism regulation. Studies have shown that this feed-back regulation leads to both hypertrophy and hyperplasia of the pancreas. A typical example refers to the prolonged administration of soybean trypsin inhibitor which can lead to invasive carcinoma⁶². Another approach includes drug modification which involves the modification of the insulin molecule itself, the hydrophobization of the insulin molecule, the preparation of monosaccharide derivatives of insulin and the binding of insulin to other proteins. However, it is desirable that insulin structure remains as much as possible as the human endogenous insulin structure. Finally, it has already been proposed to administer insulin encapsulated in liposomes. In these investigations, however, it did not appear possible to determine the amount of insulin absorbed quantitatively. The use of liposomes is moreover accompanied, as is known, by difficulties both in the preparation and in the storage of appropriate pharmaceutical forms. As well, with liposomes the oral bioavailability of insulin could not be enhanced by a meaningful amount, and in addition, carriers such as liposomes have relatively low encapsulation efficiency, and the insulin can also degrade during the encapsulation process.

So, polymeric carriers have attracted considerable and growing interest as a technology and its advancement will not only stimulate the exploration of these new oral insulin delivery systems for the therapy of Diabetes Mellitus.

Oppenheim and coworkers (1982)²⁸ have used the desolvation process to form insulin nanoparticles. These particles were effective when administered intravenously, however small quantity of insulin was delivered to systemic circulation when those particles were orally administered. Insulin was protected from its degradation in the intestine but no insulin uptake was detected.

On colonic drug delivery system, Saffran et al.⁵¹ have shown and reported on insulin-containing polymer compositions comprising an insulin-containing gelatine capsule coated by a copolymer of styrene with hydroxyethylmethacrylate, then crosslinked with a divinylbenzene azo-containing derivative. On oral administration, polymer was degraded by the action of colonic microorganisms with the release of insulin and only small amounts of insulin were able to pass through the intestinal wall. Another drawback of these compositions was that they showed a low resistance to the action of digestive enzymes. Only a very small amount of insulin passed through the intestinal wall and appeared as active insulin in the circulatory system. With the oral administration of the above composition into rats, the maximum reduction of blood glucose was 25%, far below the desirable reduction.

U.S. Pat. No. 4,849,405 proposes the embedding of insulin in a liquid, aqueous two-phase system based on a coacervate. This process has the following disadvantages: the water-soluble drug leaks out during formulation decreasing the amount of entrapped insulin. Thus it is difficult to obtain high drug content and the resulting composition has a porous structure causing an unexpected initial drug release. The insulin embedded in this coacervate should be a rapidly releasing pharmaceutical form. Some questions were not answered such as loss of activity of the insulin as a result of chemical changes during the preparation and a high loss of insulin during encapsulation, which is reflected with certainty in the preparation costs. In addition, this work described in vivo assays based on three animals (n=3). Blood glucose reduction was around 67% and 47% of basal glucose values for 5 and 10 IU/kg. In present invention, blood glucose reduction was around 70% of basal values.

Greenley et al.⁶³ immobilized insulin with a crosslinked polymer modified with inhibitor of proteolytic enzymes. The crosslinked polymer was a polyacrylic or polymethacrylic acid crosslinked with triethyleneglycoldi(meth)acrylate, and the inhibitor is represented by aprotinin-protease inhibitor. A disadvantage of this composition is a low resistance of synthesized polymer hydrogels to the action of digestive enzymes giving cause to a low activity of blood-penetrating insulin.

Another approach involved the preparation of nanocapsules by interfacial polymerisation of isobutyl-2-cyanoacrylate around a lipidic phase⁶⁴. A measurable reduction in glucose concentration of 25% was observed in fed rats when very high insulin doses were used compared to generally accepted therapeutic doses. Insulin was shown to be measurably active 20 days after the nanocapsules were administered to the animals. Thus the prolonged decrease in glycemia observed after oral administration of insulin-loaded nanocapsules was attributed to an intertissular distribution of nanocapsules, followed by the progressive degradation of the polymer to the release of insulin. However, some toxicity was associated to those polymers On the other hand, particle elimination is effected during 12-24 hours. Even if there was an adequate explanation, such a dosage level, is considered to be unacceptable for insulin, whose administration and therapeutic effect must follow closely one upon the other. It is desirable for insulin to move across the intestinal wall and enter the circulatory system along with the absorbed nutrients from food which may be ingested by the host.

U.S. Pat. No. 5,049,545 comprised insulin-containing compositions for injections where insulin is immobilized in a polymer hydrogel. The polymers useful in such compositions are starch, dextran, polyoxyethylene, polyvinylpyrrolidone, cross-linked collagen, proteins and derivatives thereof, inclusive of the inhibitors of proteolytic enzymes. These compositions displayed an increased resistance to the effect of blood proteolytic enzymes, a factor that provides an increased duration of the functioning of insulin in the bloodstream. However, the insulin-containing polymer compositions synthesized in this work do not show, enough stability to the attack of the digestive enzymes which makes their oral use impossible.

U.S. Pat. No. 5,614,219 described a process for the production of an oral administration of insulin which was associated by adsorptive charge compensation (pseudocoacervate) to an oppositely charged matrix-forming agent. The pharmaceutical form described is a layered tablet. No in vivo results were described. In our present invention, multiparticular systems as nanoparticles should have additional advantages in terms of high superficial area of intestinal absorption. Because of their sub-cellular size, submicron particles are hypothesized to enhance interfacial cellular uptake, thus achieving in a true sense an extended pharmacological drug effect.

U.S. Pat. No. 5,641,515 described a controlled release pharmaceutical formulation comprising nanoparticles formed of a biodegradable polycyanoacrylate polymer in which insulin is entrapped, the insulin being complexed to the polycyanoacrylate. In in vivo assays, a reduction in blood glucose levels to 35% of the baseline value was observed over a four hour period, indicating an oral bioavailability higher than 7% for these insulin-loaded nanoparticles.

U.S. Pat. No. 5,698,515 described an insulin-containing polymer composition intended for the oral administration of insulin, which comprises a hydrophilic polymer modified with an inhibitor of proteolytic enzyme, insulin and water, wherein the inhibitor of proteolytic enzymes is ovomucoid isolated from duck or turkey egg whites. Insulin activity in the case of the previous compositions being used orally averages 60 to 70% of the activity of initial insulin when injected.

U.S. Pat. No. 5,679,377 comprised a preparation method of zein insulin-loaded microspheres. This technique for producing particles involves the use of heat (45° C.) which may be potentially harmful to structure and consequently biological activity of insulin. As well, blood glucose profile was not stable. Significant variations on blood glucose occurred during in vivo assay.

WO9631231A described a controlled release pharmaceutical formulation comprising nanoparticles formed of a biodegradable polycyanoacrylate polymer in which insulin is entrapped, the insulin being complexed to the polycyanoacrylate. These particles are capable of releasing bioactive insulin in vivo at a slower release rate. Administration may be oral or parenteral, and for oral administration, an enteric coating may be provided to target release to the small intestine. Complexing of the insulin is achieved by the polymerisation of cyanoacrylate monomer in the presence of insulin at a low pH, preferably at pH=2. Polymers used in Patent WO9631231A are not natural occurring polymers and no toxicological studies were performed and demonstrated.

U.S. Pat. No. 5,869,103 provided insulin biodegradable microparticulate system with lactide homopolymers or copolymers of lactide and glycolide and water soluble polymers include polyethyleneglycol or copolymers. Microparticles were prepared by emulsion/solvent extraction. This works claimed oral insulin but it did not demonstrate in vivo results after oral administration of said composition.

U.S. Pat. No. 6,004,583 related to the compositions which consist of a conventional chemical compound, a protein, a peptide, or a peptide bio-mimetic incorporated within a hydrogel whose polymer structure has been chemically modified. The compositions of this invention when administered orally exhibit at least 25% of the biological efficacy of the delivered therapeutic compared to the efficacy when the therapeutic is administered by either intravenous or subcutaneous injection. Insulin activity displayed for the claimed compositions is shown to be 60% to 70% of the activity obtained when the same dosage of insulin was administered by injection whereas in present invention the insulin activity is 100% when the same dosage of insulin of dosage was administered by injection. Beside, polymers used in U.S. Pat. No. 6,004,583 are not natural occurring polymers and no toxicological studies were performed and demonstrated. As well, no drawings were described.

U.S. Pat. Nos. 6,123,965 and 6,143,211 describe a different method of nanoparticle preparation. Nanoparticles were made of poly(fumaric)/poly(lactide-co-glycolide) and the method used was phase inversion process. Polymers involved were synthetic and no questions of biodegradability were considered. This work described in vivo results but the reproducibility was questionable since the number of animals was low to consider these nanoparticles as an effective oral insulin formulation.

Patent IN187831 described alginate beads as a novel drug carrier for oral delivery of insulin. Different method was performed, well-know as emulsion/external gelation technology. This extrusion method has at least three main drawbacks⁶⁵; the first being that size reduction is limited by nozzle diameter as well the viscosity of the solution. Microparticles less than 500 μm are difficult to produce. The second drawback is that the procedure is not suitable for industrial scale-up as producing microparticles on a large scale requires a large number of nozzles to be operated simultaneously⁶⁶. Finally, microparticles tend to be teardrop-shaped due to drag forces following impact with the gelation bath⁶⁶. Particle size ranging from few millimetres to microns was obtained.

Patent WO02064115 described a homogeneous liquid formulation comprising monoglycerides, emulsifiers, organic solvents, insulin and acidic aqueous solution. This composition requires lyophilisation step to long-storage period which can be harsh step for insulin. The loading efficiency of insulin was around 50-100%. In the present invention, a drying process is not necessary, it is free of organic solvents composition and encapsulation efficiency of insulin was 85%.

### BRIEF DESCRIPTION OF THE DRAWINGS

FIG. 1 refers to insulin HPLC chromatograms: a) insulin non-encapsulated as control (insulin retention time around 5 min)*; b) insulin extracted from particles before pepsin incubation (insulin retention time around 5 min)**; c) insulin non-encapsulated as control after pepsin incubation*; d) insulin extracted from particles following pepsin incubation (insulin retention time around 5 min)**. *Note: Peak around 8 min in non-encapsulated insulin chromatogram corresponds to an additive, metacresol, which is not present in particles. **Large band between 6-9 min correspond to coating material of insulin-loaded particles.

FIG. 2 illustrates glycemia levels in diabetic rats following subcutaneous administration of insulin-loaded particles 4 IU/kg (▴, n =7), insulin solution 4 IU/kg (▪, n=6) and NaCl 0.9% (◯, n=9) in diabetic fasted rats. Before the injections, glycemia was 327±21 mg/dL. Results are expressed as means S.E.M. Statistically different from saline control: (a) p<0.05; (b) p<0.01; (c) p<0.001.

FIG. 3 shows glycemia levels following oral administration of insulin-loaded particles (▪, n=9) and empty particles (◯, n=9) in diabetic male Wistar rats fasted for 12 h. The dose of insulin was 50 IU/kg body weight. Insulin solution was used as control (Δ, n=6). Each value represents mean±SEM. Mean basal values at T0 were: 338±24 mg/dL. Statistically significant difference from free-insulin: * p<0.05.

FIG. 4 shows blood glucose levels following oral administration of insulin-loaded particles (▪, n=12) and empty particles (□, n=10) in diabetic glucose fed rats. Dose of insulin was 50 IU/kg body weight before glucose administration (2 g/kg). Each value represents mean±SEM. Mean basal value at TO was: 155±16 mg/dL. The value of glycemia before the administration of particles (i.e. T0-10 h) was 492±23 mg/dL. Statistically different from empty particles: *p<0.05.

FIG. 5 shows blood glucose levels following oral administration of insulin-loaded particles 25 IU/kg (□, n=8), insulin-loaded particles 50 IU/kg (▪, n=9), insulin-loaded particles 100 IU/kg (□, n=8), empty particles (◯, n=9) in diabetic rats fasted for 12 h. Each value represents mean±SEM. Mean basal values at T0 were: 390±21 mg/dL

FIG. 6 shows insulinemia bar plot following oral administration of insulin-loaded particles (50 IU/kg) in diabetic rats fasted for 12 h. Each value represents mean±S.E.M. (n=14). Mean basal values at time zero were: 35.0±8.2 μU/mL (t=0). Comparisons versus time zero: * p<0.05.

### SUMMARY OF INVENTION

The present invention described herein allows to solve the problems of the prior art, and provides an alternative methodology for development of an oral submicron particle system for proteins delivery using safe and natural materials. Animal studies and methodology for making said particle system which allows protein protection against proteolytic and acidic environment after oral administration are described.

### DETAILED DESCRIPTION OF THE INVENTION

Nanoparticles are defined as solid, sub-micron sized drug carriers which may or not be biodegradable^{67, 68}. The term nanoparticle is a collective name for both nanoparticles and nanocapsules. Nanoparticles have a matrix type structure. Drugs may be absorbed at the sphere surface or encapsulated within the particle. Nanocapsules are vesicular systems in which the drug is confined to a cavity consisting of an inner liquid core surrounded by a polymeric membrane⁶⁷. In this case, the active substances are usually dissolved in the inner core, but may also be adsorbed to the capsule surface⁶⁹. Nanoparticles are receiving considerable attention for the delivery of therapeutic drugs including proteins⁷⁰, antigens^{71, 72}, oligonucleotides⁷³ and genes⁷⁴⁻⁷⁶. Polymeric nanoparticles have been extensively studied as particulate carriers in the pharmaceutical and medical fields as they show promise as drug delivery systems due to controlled and sustained release properties, sub-cellular size and biocompatibility with tissue and cells¹. At the present time, size of nanoparticles should be considered lower than 100 nanometers while the size of submicron particles is lower than 1 micron. Generally, submicron particles have relatively higher intracellular uptake compared to microparticles and are available to a wider range of biological targets due to their small size and relative mobility⁷⁶. Several methods to prepare submicron particles have been developed during the last two decades and are well known in the art, classified according to whether the particle formation involves a polymerization reaction, or from a macromolecule or preformed polymer⁷⁷. Synthetic biodegradable polymers such as polyalkylcyanoacrylates (PACA), poly-L-glycolic acid (PLGA) and polyanhydride have received considerable attention. Despite the interest, toxicological problems may limit their applicability. In addition, these materials often present limitations for the administration of hydrophilic molecules such as proteins and peptide since the polymers are mostly hydrophobic, whereas proteins and peptides are often hydrophilic. Therefore, the preparation of submicron particles using more hydrophilic and naturally occurring materials has been explored. Methods using natural polymers and proteins are a good alternative for the encapsulation of protein and peptide drugs, because they can be formulated under gentle conditions and the encapsulation materials are generally biocompatible. In the present invention, submicron particles are produced under mild processing conditions by using emulsion dispersion/triggered gelation technology and coated by two-layer coating material herein described.

The use of this submicron particle system allows that the immobilized protein passes through the digestive tract, absorbed through the intestinal mucosa and enters the circulatory system where it can exerts therapeutic effect. Upon arrival at a target site a large portion of the drug may have already been released, leaving only a small portion of the drug for local delivery, or may pass through the site unreleased to a significant degree.

The low stomach pH and presence of gastric enzymes have led to the development of enteric coating. Coating is done by albumin, which protects insulin from inactivation by gastric enzymes. As well, an additional re-inforcement of the matrix using dextran sulfate was done to prevent insulin release at low pH. Finally, this invention relates another strategy. Mucoadhesive properties and insulin stability were improved by coating the resultant nanoparticles with chitosan and polyethylene glycol.

Since this invention described insulin-loaded particles with a unimodal size distribution where 90% had diameter less than 1842 nm and 50% were less than 812 nm, an additional benefit is demonstrated with present invention. It has been observed that a greater number of nanoparticles cross the epithelium than do microparticles. Moreover, these particles with size range less than 10 μm can be captured by lymphatic system especially M-cells of Peyer's patches. Targeting the Peyer's patches in a particular segment of the small intestine can be useful in limiting destructive side reactions. Capillary and lymphatic vessels are very permeable to lipid-soluble compounds and low molecular weight moieties. However, this absorption is size-dependent. Particle uptake via lymphatic system after oral delivery increases exponentially as particle size decrease from 10 μm into the submicron range¹. Macromolecules, such as insulin, could be absorbed through Peyer's patches, which occur equally throughout all segments of the small intestine. Peyer's patches are most prevalent in young individuals. Generally, the most serious diabetes form is juvenile diabetes and it is well-know as diabetes type 1. This diabetes form requires daily insulin injection and there is not another therapy available in the market for those patients. However, as Peyer's patches are characterized by age-related disappearance; they provide a target site for absorption until middle age.

In the drug delivery field, submicron and nanoparticles have demonstrated numerous advantages over conventional formulations; however, most techniques available for producing them involve the use of organic solvents, heat or vigorous agitation which are potentially harmful to structure and consequently biological activity of proteins. As used herein, the term “nanoparticles” refers to particles having a diameter of preferably between 20 nm and about 100 nm. while the term “submicron particles” refers to particles having a diameter of preferably lower than 1 micron.

Because of their sub-cellular size, submicron particles are hypothesized to enhance interfacial cellular uptake, thus achieving in a true sense an extended pharmacological drug effect.

In the present invention, submicron particles are produced under mild processing conditions by using emulsion dispersion/triggered gelation technology and coated by two-layer coating material herein described. As used herein, the term “dispersion” refers to the distribution of particles throughout a medium, such as solvent and the last one; the term “solvent” refers to any liquid substance that is capable of dissolving, dispersing, or suspending one or more other substances.

A wide variety of water-soluble therapeutic agents, such as proteins, peptides and nucleic acids, can be encapsulated using this method and composition of the present invention. The term “agent” refers to water-soluble solute including proteins and peptides for administration to a subject, such as a human, animal or other mammal. While selected based upon the intended application or therapy, the agent is typically a therapeutic water-soluble drug, the efficacy of which can be improved or optimized when administered orally with a programmable, extended release pharmacokinetic profile, as is accorded by the present invention. The basic approach involves suspending the protein to be encapsulated in a biocompatible immobilizing containing a water soluble substance, polymer, that can be made insoluble in water, that is, gelled to provide a protective microenvironment for the protein. The term “polymer” includes any film forming polymer of natural, synthetic, or semi synthetic origin, and may be biodegradable or not. Polymers prepared from renewable natural resources have become increasingly important because of their low cost, ready availability, water-solubility, biocompatibility, biodegradability and gel forming ability.

In the present invention the immobilizing agent is a naturally occurring polysaccharide. Alginate in sodium form salt is the preferred immobilizing agent. Alginate forms stable reversible gels in the presence of multivalent cations under gentle formulation conditions at room temperature. Chemically, carboxylic groups of alginate react with multivalent cations and form a polymer network, well-know as egg-box structure. Alginate polymer is inexpensive, natural, biodegradable, non-toxic, widely available as food or medical grade material and biocompatible. Alginate also has several unique properties that have enabled its use as a matrix for entrapment and/or delivery of a variety of proteins and cells. Over the last decades, more suppliers of alginates are appearing in the market place; the quality of the polymer is improving, and alginates are now being sold partially or fully characterized in terms of its chemical and physicochemical properties. In present invention, alginate presents a molecular weight ranging from 250 to 300 kDa, low guluronic content and low viscosity (2% w/v with 250 cps).

Dextran sulfate was used as adjuvant in present formulation due to its permanent negative charge (sulfate groups). Dextran sulfate increased the negative microenvironment for insulin. The two polyanions, alginate and dextran sulfate, provide both pH-sensitive (carboxylate) and permanently charged (sulfate) groups and avoid the release of positive charged insulin at low pH in the stomach.

The selection of a suitable calcium vector for internal gelation of alginate depends on the range of initial to final pH values desired. Over the pH range of interest, the concentration of free calcium must be very low initially with rapid release of calcium while reducing pH. A pKa value of the anions in the working range (6.5 to 7.5) is optimal for peptide immobilization.

Surfactants or emulsifying agents may also be used in emulsion systems to lower the interface tension between the water and oil phases and to make the dispersion of viscous alginate solution into the oil easier. In this particular case, Span 80® demonstrates great stability and it easily avoids the coalescence phenomenon. According to the present invention, the mineral oil used in the preparation of the particles is paraffin oil mainly due to its high viscosity.

The selection of a suitable oil-soluble organic acid is significant. In the present invention, oil-soluble acid was glacial acetic acid. The oil-soluble organic acid is initially added in the oil which has the effect of immediately partitioning into the aqueous phase, thus instantaneously lowering the pH of the dispersed droplet, solubilizing crystalline calcium, triggering a rapid gelation.

This invention herein decribes a new coating process. The first layer or the coat of said pharmaceutical form is then built up to protect insulin from gastric enzymes. The second layer, on the other hand, is made to promote the mucoadhesion to intestinal mucosa, to improve the half-live of insulin and finally to increase residence time along the intestine.

Two-layer coating were herein described and include a primary coating which comprises a blend at pH 4.5 rich in calcium ion, chitosan in acetate salt form with molecular weight around 50 kDa and polyethylene glycol (PEG) with molecular weight of 4 kDa. Mucoadhesive properties of chitosan may have increased the probability that the nanoparticles adhere and be absorbed by enterocytes. The term “mucoadhesive” refers to the capacity of particles to adhere to the mucosal layer which lines the entire surface of the small and large intestine. Chitosan and PEG are non-toxic and generally harmless to proteins and cells. Chitosan salts can bind strongly to negatively charged materials such as cell surfaces and mucus. Mucus contains mucins that have different chemical constitutions but some contain significant proportions of sialic acid. At physiological pH, sialic acid carries a net negative charge and, as a consequence, mucin and chitosan can demonstrate strong electrostatic interaction when in solution. Chitosan also has the effect of transiently opening the tight junctions in mucosal membranes. Both the bioadhesive characteristics of the chitosan and its transient effect on tight junctions could lead to an improved pharmacological response of protein drug. Of the numerous applications of PEG can be mentioned: as covering-agent of the surface of nanoparticles to increase their residence time in the circulation, and as covalent attachment-agent to proteins to obtain conjugates which are still biologically active but no longer immunogenic and antigenic; such PEG-protein adducts having been approved for parenteral use in humans. In addition, coating conventional nanoparticles with PEG to obtain a long-circulating carrier has now been used as a standard strategy for drug targeting in vivo.

Second coating includes an aqueous solution of bovine serum albumin at pH 5.1. We hypothesized that albumin becomes the degradative target for gastric enzymes (pepsin). Without albumin, positively charged particles reach the intestine and strongly interact with intestinal mucosa. Indeed sialic acid residues of mucus are negatively charged at physiological pH⁷⁸ and without albumin, insulin-loaded nanoparticles have now a positive charge. This may increase the residence time next to the absorption surface of the gastrointestinal tract and create a drug concentration gradient toward the blood. Besides the mucoadhesive properties of this novel insulin carrier, small particle size allows significant particle diffusion by paracellular mechanism. Albumin and chitosan-PEG chains strongly influence the mesh size of the hydrogel networks⁷⁹, forming an additional capsule shell, made up of an interpenetrating network near the surface of the core particle, preventing pepsin attack.

Thus, a first object of the invention is an oral submicron particle delivery system for proteins to be immobilized which comprises:
a. a core comprising said protein to be immobilized, a naturally occuring immobilizing agent, an adjuvant, and an immobilizing agent crosslinker to obtain gelled submicron particles by an emulsification-based method;
b. a primary coating material surrounding said core which comprises a blend of hydrophilic, natural and biodegradable polymers;
c. a secondary coating material surrounding said primary coating wherein said secondary coating material comprises protein material.

In a preferred embodiment said protein comprises unmodified human insulin as a drug to be immobilized in said immobilizing agent.

The immobilizing agent comprises, usually, a naturally-occuring polysaccharide, preferably a sodium alginate, a polyanionic polymer at pH 4.5, which gels in presence of divalent ions.

The preferred immobilizing agent crosslinker is calcium released from calcium complex, namely calcium carbonate.

It is a second object of the invention an emulsification-based process for production of an oral submicron particle delivery system for proteins, of claims 1 to 7, which comprises:
a. providing an aqueous phase, internal phase, containing an immobilizing agent, an adjuvant, immoblized protein and an immobilizing agent crosslinker to cause gelation of said immobilizing agent,
b. contacting said internal phase with a hydrophobic liquid, external phase, under mild conditions leading to formation of droplets of said internal phase in said external phase, and
c. adding an oil-soluble organic acid to mixture obtained in step b) to convert said droplets into gel particles,
d. recovering the resultant gelled submicron particles,
e. primary coating the recovered gelled submicron particles using a blend of hydrophilic polymers with high calcium levels, and
f. coating the primary coated submicron particles in step e) using protein coating material.

This process normally comprises introducing protein to be immobilized into said immobilizing agent and the adjuvant so as to obtain solid containing said protein, said immobilizing agent and said adjuvant.

The preferred immobilizing agent crosslinker, as referred to above, is calcium released from calcium complex.

In addition, the process may comprise adding a pH-decreasing compound, which dissolve the calcium complex, to said mixture b).

Usually said adjuvant comprises dextran sulfate said hydrophobic liquid is paraffin oil and said oil-soluble acid is acetic acid.

The process may comprise, additionally, removing residual oil using partition phases, centrifuge and thereby to produce a colloidal suspension of particles.

The blend of hydrophilic polymers comprises, preferably, chitosan at a concentration of about 0.015% to 0.15% (w/w), polyethyleneglycol at a concentration of about 0.0375% to 0.3% (w/w) and calcium chloride at a concentration of about 1.5% (w/w) at pH 4.5 and the protein coating material is albumin at a concentration of about 0.5% to 1.5% (w/w) at pH 5.1.

The following Examples are intended to illustrate the invention, without any limitation in the scope of the invention, only defined by the appended claims.

### Example 1

### Method for Producing Formulation Media

The following methodology will produce 50 ml of media:
(1) mix 40 ml of distilled water, 1 gram of sodium alginate to yield at 2% solution of alginate and 0.0375 g of dextran sulfate to yield at 0.75% using a orbital shaker (100 rpm, 8 h);
(2) following stationary deaeration of solution in step 1 for 1 h), insulin was added and dissolved (100 IU/mL, 10 mL) and add 10 mL of insulin (35 mg) to aqueous alginate-dextran solution;
(3) add 3.5 mL of a 5% (w/v) calcium carbonate to (2) and mix for a further 2 minutes.

### Example 2

### Formation of Alginate-Dextran-In-Oil Emulsion

The following technique is used to emulsify immobilizant media in oil media with surfactants at 1.5% v/v, Span 80®, under high speed homogenization. The oil is mixed with the immobilizing agent produced in EXAMPLE 1 in proportions ranging from 50:50 and mixed at 1600 rpm under impeller stirring for 15 minutes.

### Example 3

### Production of Alginate-Dextran-In-Oil Dispersion

In a more preferred mode, the reactor is filled with oily phase. The reactor must be appropriate for a 100 mL batch mixer. The apparatus used to produce an appropriate dispersion can be a regular mixing device with a capacity to high speed rates. Relative to the most appropriate impeller, this invention employs three standard baffles commonly known as a marine type impeller. This invention uses a high-speed digital laboratory mixer with maximum speed of 2000 rpm:
(1) 50 mL of the solution prepared via the method outlined in Example 1 is mixed with the oil phase in a ratio of 50:50;
(2) 50 mL of mineral oil containing 1.5% (v/v) of emulsifier (Span 80®, sorbitan monooleate) is placed in the reactor and the impeller speed set at 1600 rpm;
(3) the solution prepared in step 1 is added to the reactor while stirring is maintained. Stirring is continued for 15 minutes to allow the dispersion to properly form;
(4) while still stirring, 20 mL of mineral oil containing 0.3 mL glacial acetic acid is then added to the reactor;
(5) after 60 minutes, the impeller speed rate is reduced to 200 rpm and 70 mL of acetate buffer solution at pH 4.5 prepared according to USP XXVII, 15 mL of acetone, 10 mL isopropanol and 5 mL of hexane is added to gelled submicron particles for 2 minutes;
(6) resultant gelled submicron particles are stored at 4° C. during 24 hours;
(7) oil-dispersed alginate particles are recovered by using the washing medium coupled with centrifugation at 12500×g;
(8) recovered alginate particles are stored at 4° C.

### Example 4

### Method for Producing Primary Coating Medium

The following methodology will produce 100 mL of media:
(1) mix 100 mL of distilled water, 0.03 gram of acetate chitosan to yield at 0.03% solution of chitosan, 1 mL of glacial acetic acid, 0.15 gram of polyethylene glycol to yield at 0.15% of PEG and 1.5 gram of chloride calcium using a magnetic shaker (100 rpm, 60 min);
(2) adjust the final pH to 4.5.

### Example 5

### Primary Coating of Insulin-Loaded Submicron Particles

(a) Recovered submicron alginate particles in EXAMPLE 3 are coated with primary coating medium during 30 min under magnetic stirring (100 rpm);
(b) then, unreacted polymers were removed by vaccum.

### Example 6

### Method for Producing Secondary Coating Medium

The following methodology will produce 100 mL of media:
(a) mix 100 mL of distilled water with 1 gram of bovine seric albumin using a magnetic shaker (100 rpm, 60 min);
(b) adjust the final pH to 5.1.

### Example 7

### Secondary Coating of Insulin-Loaded Submicron Particles

a) Coated submicron alginate particles in EXAMPLE 6 are now coated with secondary coating medium during 30 min under magnetic stirring (100 rpm);
b) then, unreacted polymer was removed by vaccum.

### Size Characterization

Size distribution analysis was performed by laser diffraction spectrometry using a Coulter LS130 granulometer (Beckman Coulter Inc., Fullerton, Calif.). Mean diameters of aqueous suspensions were determined in triplicate and size distribution was represented by number. Insulin-particles showed a unimodal size distribution where 80% had diameter less than 1842 nm, and 50% were less than 812 nm.

### Determination of Encapsulation Efficiency of Insulin

Encapsulation efficiency was determined after chitosan/PEG/BSA-coating by analysing filtrate, wash and dehydration solutions using insulin enzyme linked-immuno-sorbent assay insulin kit (ELISA, Mercodia, Sweden) at 450 nm. Encapsulation efficiency (%) determined by insulin released as percentage of initial amount used in formulation. Encapsulation efficiency of insulin within the particles was high at 85±4% mainly due to the partition of insulin into the aqueous medium.

Insulin Molecular Integrity after Pepsin Incubation

Insulin (μg insulin per mL particles) molecular integrity was evaluated by HPLC after particles matrix dissolution with citrate (55 mM). Insulin was quantified initially, and after 2 h incubation in simulated gastric fluid containing pepsin at pH 1.2 (USP XXVIII, pepsin at 2080 Units/mg protein) in a shaking water bath at 37° C. and 100 rpm. Particles were recovered by centrifugation and transferred to citrate solution with stirring for 1 h, then aliquots collected, centrifuged and analyzed by HPLC. Non-encapsulated insulin served as reference and assays were conducted in triplicate. Enzyme resistance was calculated by insulin content after enzyme incubation as percentage of initial insulin content.

Insulin in alginate-dextran/chitosan-PEG/albumin submicron particles was fully retained, and protected from pepsin attack in simulated stomach fluid—likely due to alginate polymer forming compact “acid-gel” structure reducing permeability, and albumin serving as sacrificial target for low molecular weight protease. Albumin is long-lived in vivo⁸⁰ and may protect insulin from proteolysis prior to its detachment from the particulate matrix. Albumin may act as degradative target of pepsin. Compact submicron particles may also stabilize insulin from acid attack. At low pH, alginate contracts due to alginic acid precipitation, resulting in a compact and impermeable matrix. Ca2+ is released, potentially destabilizing polymer at subsequent neutral pH. At this point, the matrix swells, promoting insulin release. As well, at neutral pH, both alginate and insulin are negatively charged and electrostatic repulsion may promote release, but presence of chitosan membrane may have a stabilizing-retentive effect.

### Assay Procedure

Samples were protein assayed using HPLC where mobile phase was water (A): acetonitrile (B) with 0.04% trifluoroacetic acid with linear gradient B 30% to 40% over 5 min, flow rate 1.2 mL/min at 25° C.

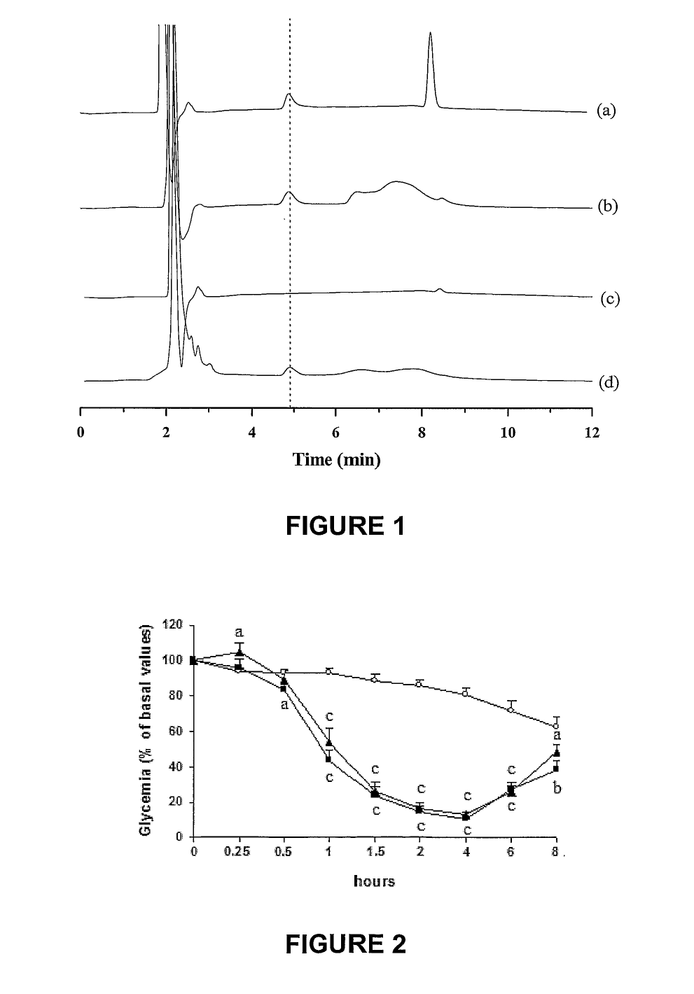

Insulin-loaded submicron particles were exposed to pepsin, and extracted insulin run on HPLC. Particles released intact insulin, with peak appearing consistent with non-treated control, as seen in FIG. 1. Free insulin was subject to pepsin attack, with peak having been eliminated. Moreover, insulin transformation products were not detected which suggested the maintenance of insulin stability after enzyme incubation.

### Glycemic Response of Diabetic Rats in Response to Insulin Particles Dosage

Male Wistar rats (250 g) were housed in a 12-12-h light-dark cycle, constant temperature environment of 22° C., relative humidity 55% and allowed free access to water and food during acclimatization. Animals received standard laboratory chow diet (UAR, Villemoisson-sur-Orge, France) and tap water, available ad libitum. All treatments began between 8.00 and 9.00 h. Animal procedures were reviewed and approved by the committee for animal research according the Institutional European Guidelines (n° 86/609). To minimize the diurnal variance of blood glucose, all experiments were performed in the morning⁸¹. Diabetes was induced with intravenous injection of 65 mg/kg streptozotocin in citrate buffer at pH 4.5 as previously described³⁰. Ten days after the treatment, rats with frequent urination, loss of weight and fasting blood glucose levels higher than 350 mg/dL were included in experiments. Blood glucose levels were determined using a glucometer (Accuchek Go, Roche, France).

In order to verify that the particles contained bioactive insulin, submicron particles were injected subcutaneously to fasted diabetic rats (4 IU/kg body weight). Free insulin (4 IU/kg) was administered as control. Glycemia in blood samples withdrawn from the tail vein was measured before insulin injection and at intervals to 8 h. Rats were fasted 12 h prior to dosing, during the experiment and fed thereafter.

Oral dosage was 50 IU/kg of animal body weight. Submicron particles with and without insulin were administrated orally through a tube which was attached to a hypodermic syringe and approximately 2 mL in the aqueous dispersion medium was administered. Pharmacological availability of peroral delivered insulin was determined based on 100% availability of the particles suspension administered subcutaneously at a dose of 4 IU/kg. Serum glucose time course was plotted, and the area below the 100% cut-off line determined using the trapezoidal method during 0-8 h. Insulin concentration was also evaluated using insulin radioimmunoassay (RIA kit, CIS Bio International, Gif-Sur-Yvette Cedex, France). Insulin versus time curve was plotted and the relative bioavailability after intragastric administration was then calculated.

The effect of insulin-loaded submicron particles on postprandial rise in blood glucose was tested. Empty or insulin-loaded submicron particles were given orally 10 h before the glucose gavage (50 IU/kg), and glycemia measured. Then, rats were given 2 g/kg glucose orally in water dispersion through a gastric tube at 0 min, 20, 40, 60, 90, 120 and 180 min and glycemia determined.

In a single administration study, dose-response effect was also studied. Insulin doses of 25, 50 and 100 IU/kg were administrated by gavage and hypoglycaemic effect estimated from blood glucose levels.

Insulinemia levels were also determined after oral dosage of insulin and free-insulin submicron particles. Rats were anaesthetized with ketamine and xylazine and blood was withdrawn into EDTA tubes, centrifuged, and the plasma stored at −15° C., representing initial insulinemia levels. Oral insulin dosage was 50 IU/kg. Plasma immunoreactive insulin was measured by radioimmunometric assay (Insulin-CT kit from CIS Bio international, GIF-Sur-Yvette Cedex, France) for up to 12 h.

Statistical Analysis of In vivo Data

Results were expressed as means±standard errors of means (S.E.M.). For group comparison, an analysis of variance (ANOVA) with a one-way layout was applied. Significant differences in mean values were evaluated by a Student's t-test. For multiple comparison group tests, a Bonferroni or a Dunnett multicomparison test was applied, using Instat 2.00 Macintosh software (Graph Pad Software, San Diego, Calif.). The differences were considered significant when P<0.05.

### Results

### Efficacy of Insulin-Loaded Submicron Particles on Diabetic Rats

Blood glucose profiles following subcutaneous injection of insulin-loaded submicron particles and non-encapsulated insulin to diabetic rats (4 IU/kg) are illustrated in FIG. 2. Both loaded-particles and free insulin control promoted a rapid and intense decrease of glycemia with glucose levels approaching 13% after 4 h. There were no statistical differences between insulin groups.

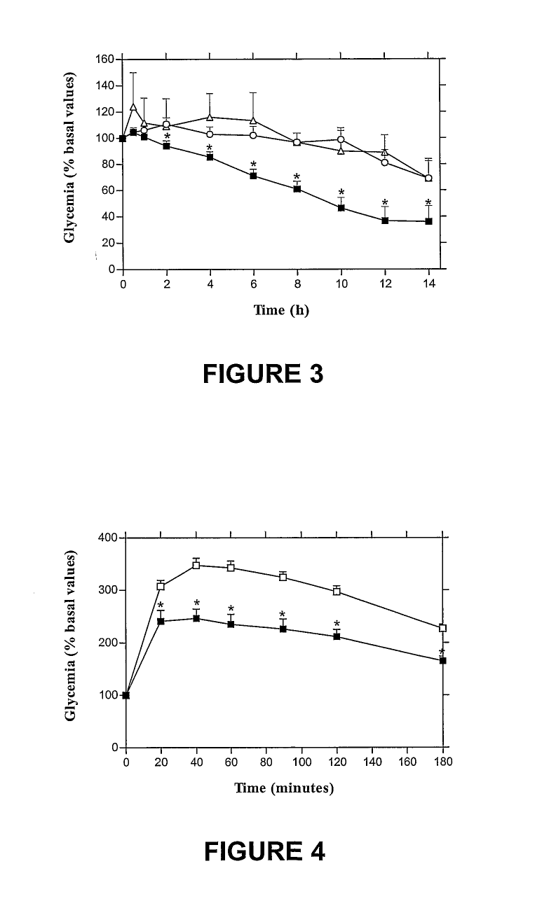

Diabetic fasted rates were dosed orally with insulin-loaded submicron particles, using empty particles and insulin solution as controls. Insulin-loaded submicron particles suppressed the initial rise in blood glucose levels as compared with empty particles as illustrated in FIG. 3. Insulin-loaded submicron particles significantly reduced blood glucose up to 14 h. Hypoglycaemic effect was not observed with insulin solution, demonstrating that the insulin cannot be absorbed enough by the oral route in the absence of a suitable carrier. As well, a hypoglycaemic effect was not observed with empty particles.

Postprandial rise in blood glucose after high level of glucose administration was assayed (2 g glucose/kg). When diabetic rats were dosed orally with insulin-loaded particles, glycemia also increased as seen in FIG. 4 but less so than the controls which were dosed empty particles. Insulin-loaded particles reduced postprandial hyperglycemia during 180 min. Differences in response between empty and insulin-loaded particles treatments were significant statistically. Consequently, insulin-loaded particles improved the response to glucose challenge for an extended period.

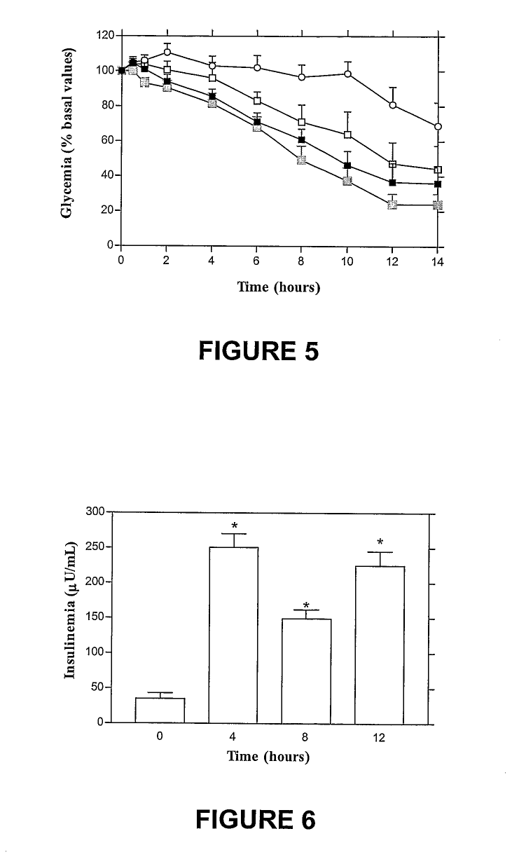

Fasted diabetic rats were treated with single dose of 25, 50, or 100 IU/kg, and the glucose response monitored. Insulin-loaded particles decreased glycemia in a dose-dependent manner by comparison with rats treated with empty particles as shown in FIG. 5, showing a strong hypoglycemic effect during 14 h. The maximum effect was observed at 12 h with strong activity. Between 100 and 50 IU/kg, the differences observed were not statistically significant. Pharmacological availability values were calculated and summarized in Table 1.

TABLE 1
Pharmacological availability of insulin-loaded submicron
particles administered to diabetic rats. Each value
represents mean ± SEM.

| | | | Pharmacological | |
|---|---|---|---|---|
| | Dose | Area under | availability at | tₘₐₓ |
| Route | (IU/kg) | the curve | 8 h (%) | (h) |
| Subcutaneous | 4 | 265 ± 18 | 100 | 4 |
| Oral | 25 | 696 ± 43 | 42 | 14 |
| | 50 | 670 ± 31 | 21 | 14 |
| | 100 | 630 ± 36 | 10 | 14 |

Values ranged from 10 to 42% of subcutaneous administration for doses 100 to 25 IU/kg, respectively.

Insulinemia increased with a maximal effect 4 h after gavage by factor of seven of basal value as illustrated in FIG. 6. Initial insulin concentration was 35.0±8.2 μU/mL (t=0) but increased to 250.8±19.4 μU/mL (p<0.001) at 4 h. At 8 hours, plasma insulin level was still high 149.4±12.7 μU/mL (p<0.001) and increased again at 12 hours, being 225.1±20.2 μU/mL (p<0.001). These results clearly showed that insulin absorption was markedly enhanced by the action of insulin-loaded submicron particles. This quantification method is highly specific for human insulin as cross-reaction with rat insulin is less than 0.03%. Over the assay, insulin concentration values were higher than basal values. Insulinemia versus time plot was traced. AUC_{0-8 h} of orally administered particles was 5256. The corresponding relative bioavailability of insulin was calculated from the areas under the curves (trapezoidal method) and the doses administered orally and subcutaneously were 34% as shown in Table 2.

TABLE 2
Oral relative bioavailability of insulin-loaded submicron
particles administered to diabetic rats, n = 14.

| | | Area under | Oral |
|---|---|---|---|
| | Dose | the curve | bioavailability |
| Route | (IU/kg) | (8 h) | (%) |
| Subcutaneous | 10 | 3061 | |
| Oral | 50 | 5256 | 34 |

These results clearly showed that insulin absorption was markedly enhanced by the action of insulin particles.

### REFERENCES

### Patent Documents

U.S. Pat. No. 4,849,405 July 1989 Ecanow
U.S. Pat. No. 5,049,545 September 1991 Lobermann et al.
U.S. Pat. No. 5,614,219 March 1997 Wunderlich et al.
U.S. Pat. No. 5,641,515 June 1997 Ramtoola
U.S. Pat. No. 5,698,515 December 1997 Plate et al.
U.S. Pat. No. 5,869,103 February 1999 Yeh et al.
U.S. Pat. No. 6,004,583 December 1999 Plate et al.
U.S. Pat. No. 6,123,965 September 2000 Jacob et al.
U.S. Pat. No. 6,143,211 November 2000 Mathiowitz et al.
WO9631231 October 1996 Ramtoola Zebunnisa
IN18731 June 2002 Chandy et al.
WO02064115 August 2002 Jung Hye Seon et al.

### Non-Patent Documents

1. Reis, C. P., Neufeld, R. J., Ribeiro, A. J. & Veiga, F. Nanoencapsulation II. Biomedical applications and current status of peptide and protein nanoparticulate delivery systems. Nanomedicine: Nanotechnology, Biology and Medicine 2, 53-65 (2006).
2. Kang, F. & Singh, J. In vitro release of insulin and biocompatibility of in situ forming gel systems. International Journal of Pharmaceutics 304, 83-90 (2005).
3. Owens, D. R., Zinman, B. & Bolli, G. Alternatives routes of insulin delivery. Diabetes 20, 886-898 (2003).
4. Damgé, C., Vranckx, H., Balschmidt, P. & Couvreur, P. Poly(alkylcyanoacrylate) nanospheres for oral administration of insulin. Journal of Pharmaceutical Sciences 86, 1403-1409 (1997).
5. Morishita, I., Morishita, M., Takayama, K., Machida, Y. & Nagai, T. Hypoglycemic effect of novel oral microspheres of insulin with protease inhibitor in normal and diabetic rats. International Journal of Pharmaceutics 78, 9-16 (1992a).
6. Fujii, S., Yokoyama, T., Ikegaya, K., Sato, F. Yokoo, N. Promoting effect of the new chymotrypsin inhibitor FK-448 on the intestinal absorption of insulin in rats and dogs. Journal of Pharmacy and Pharmacology 37, 545-549 (1985).
7. Morishita, I., Morishita, M., Takayama, K., Machida, Y. & Nagai, T. Enteral insulin delivery by microspheres in 3 different formulations using Eudragit L100 and S100. International Journal of Pharmaceutics 91, 29-37 (1993):
8. Morishita, M., Morishita, I., Takayama, K., Machida, Y. & Nagai, T. Site-Dependent effect of Aprotinin, Sodium Caprate, Na2EDTA and Sodium Glycholate on intestinal absorption of insulin. Biology Pharmaceutical Bulletin 16, 68-72 (1993).
9. Yamamoto, A. et al. Effects of various protease inhibitors on the intestinal absorption and degradation of insulin in rats. Pharmaceutical Research 11, 1496-1500 (1994).
10. Mesiha, M., Plakogiannis, F. & Vejosoth, S. Enhanced oral absorption of insulin from desolvated fatty-acid sodium glycocholate emulsions. International Journal of Pharmaceutics 111, 213-216 (1994).
11. Fix, J. A. Absorption enhancing agents for the GI system. Journal of Controlled Release 6, 151-156 (1987).
12. Shao, Z., Li, Y., Krishnamoorthy, R., Chermak, T. & Mitra, A. K. Differential effects of anionic, cationic, nonionic, and physiologic surfactants on the dissociation, alpha-chymotryptic degradation, and enteral absorption of insulin hexamers. Pharmaceutical Research 10, 243-251 (1993).
13. Uchiyama, T. et al. Enhanced permeability of insulin across the rat intestinal membrane by various absorption enhancers: their intestinal mucosal toxocity and absorption-enhancing mechanism of n-Lauryl-B-D-maltopyranoside. Journal of Pharmacy and Pharmacology 51, 1241-1250 (1999).
14. Touitou, E. & Rubinstein, A. Targeted enteral delivery of insulin to rats. International Journal of Pharmaceutics 30, 95-99 (1986).
15. Shao, Z., Li, Y., Chermak, T. & Mitra, A. K. Cyclodextrins as mucosal absorption promoters of insulin. Part 2. Effects of beta-cyclodextrin derivatives on alpha-chymotryptic degradation and enteral absorption of insulin in rats. Pharmaceutical Research 11, 1174-1179 (1994).
16. Schilling, R. J. & Mitra, A. K. Intestinal mucosal transport of insulin. International Journal of Pharmaceutics 62, 53-64 (1990).
17. Scott Moncrieff, J. C., Shao, Z. & Mitra, A. K. Enhancement of intestinal insulin absorption by bile salt-fatty acid mixed micelles in dogs. Journal of Pharmaceutical Sciences 83, 1465-1469 (1994).
18. Fasano, A. & Uzzau, S. Modulation of intestinal tight junctions by zonula occuldens toxin permits enteral administration of insulin and other macromolecules in an animal model. Journal of Clinical Investigation 99, 1158-1164 (1997).
19. Asada, H. & al., e. Absorption characteristics of chemically modified-insulin derivatives with various fatty acids in the small and large intestine. Journal of Pharmaceutical Sciences 84, 682-687 (1995).
20. Hashizume, M. et al. Improvement of large intestinal absorption of insulin by chemical modification with palmitic acid in rats. Journal of Pharmacy and Pharmacology 44, 555-559 (1992).
21. Asada, H. et al. Absorption characteristics of chemically modified-insulin derivatives with various fatty acids in the small and large intestine. Journal of Pharmaceutical Sciences 84, 682-687 (1995).
22. Hashimoto, T., Nomoto, M., Komatsu, K., Haga, M. & Hayashi, M. Improvement of intestinal absorption of peptides: adsorption of Bl-Phe monoglucosylated insulin to rat intestinal brush-border membrane vesicles. European Journal of Pharmaceutical and Biopharmaceutics 50, 197-204 (2000).
23. Still, J. G. Development of oral insulin: progress and current status. Diabetes Metabolism Research and Reviews 18, S29-S37 (2002).
24. Hinds, K. D. et al. PEGylated insulin in PLGA microparticles. In vivo and in vitro analysis. Journal of Controlled Release 104, 447-460 (2005).
25. Calceti, P., Salmaso, S., Walker, G. & Bernkop-Schnurch, A. Development and in vivo evaluation of an oral insulin-PEG delivery system. European Journal of Pharmaceutical Sciences (2004).
26. Morcol., T., Nagappan, P., Nerenbaum, L., Mitchell, A. & Bell, S. J. D. Calcium phosphate-PEG-insulin-casein (CAPIC) particles as oral delivery systems for insulin. International Journal of Pharmaceutics 277, 91-97 (2004).
27. Lewis, D., Schmidt, P. & Hinds, K. US; 2004, U.S. Pat. No. 6,706,289.
28. Oppenheim, R. C., Stewart, N. F., Gordon, L. & Patel, H. M. Production and evaluation of orally administered insulin nanoparticles. Drug Development and Industrial Pharmacy 8, 531-546 (1982).
29. Lowe, P. J. & Temple, C. S. Calcitonin and insulin in isobutylcyanoacrylate nanocapsules: protection against proteases and effect on intestinal absorption in rats. Journal of Pharmacy and Pharmacology 46, 547-552 (1994).
30. Damgé, C., Michel, C., Aprahamian, M. & Couvreur, P. New approach for oral administration of insulin with polyalkylcyanoacrylate nanocapsules as drug carrier. Diabetes 37, 246-251 (1988).
31. Aboubakar, M. Physico-chemical characterization of insulin-loaded poly(isobutylcyanoacrylate)nanocapsules obtained by interfacial polymerization. International Journal of Pharmaceutics 183, 63-66 (1999).
32. Pan, Y. et al. Bioadhesive polysaccharide in protein delivery system: chitosan nanoparticles improve the intestinal absorption of insulin in vivo. International Journal of Pharmaceutics 249, 139-147 (2002).
33. Radwan, M. A. Enhancement of absorption of insulin-loaded polyisobutylcyanoacrylate nanospheres by sodium cholate after oral and subcutaneous administration in diabetic rats. Drug Development and Industrial Pharmacy 27, 981-989 (2001).
34. Watnasirichaikul, S., Rades, T. & Tucker, I. G. In vitro release and oral bioactivity of insulin in diabetic rats using nanocapsules dispersed in biocompatible microemulsion. Journal of Pharmacy and Pharmacology 54, 473-480 (2002).
35. Pinto-Alphandary, H. Visualization of insulin-loaded nanocapsules: in vitro and in vivo studies after oral administration to rats. Pharmaceutical Research 20 7, 1071-1084 (2003).
36. Cui, F., Mang, L., Zheng, J. & Kawashima, Y. A study of insulin-chitosan complex nanoparticles used for oral administration. Journal Drug Delivery Sciences Technology 14, 435-439 (2004).
37. Sajeesh, S. & Sharma, C. P. Cyclodextrin-insulin complex encapsulated polymethacrylic acid based nanoparticles for oral insulin delivery. International Journal of Pharmaceutics In Press, Corrected Proof (2006).
38. Zhang, N., Ping, Q. N., Huang, G. H. & Xu, W. F. Investigation of lectin-modified insulin liposomes as carriers for oral administration. International Journal of Pharmaceutics 294, 47-259 (2005).
39. Patel, H. M. & Ryman, B. E. Oral administration of insulin by encapsulation within liposomes. FEBS Letters 62, 60-63 (1976).
40. Iwanaga, K. et al. Oral delivery of insulin by using surface coating liposomes: improvement of stability of insulin in GI tract. International Journal of Pharmaceutics 157, 73-80 (1997).
41. Iwanaga, K. et al. Application of surface-coated liposomes for oral delivery of peptide: effects of coating the liposome's surface on the GI transit of insulin. Journal of Pharmaceutical Sciences 88, 248-252 (1999).
42. Kim, A., Yun, M. O., Oh, Y. K., Ahn, W. S. & Kim, C. K. Pharmacodynamics of insulin in polyethylene glycol-coated liposomes. International Journal of Pharmaceutics 180, 75-81 (1999).
43. Wu Z H, P. Q., Wei Y, Lai J M. Hypoglycemic efficacy of chitosan-coated insulin liposomes after oral administration in mice. Acta Pharmacology Sin. 25, 966-972 (2004).
44. Matsuzawa, A., Morishita, M., Takayama, K. & Nagai, T. Absorption of insulin using water-in-oil-in-water emulsion from enteral loop in rats. Biology Pharmaceutical Bulletin 18, 1718-1723 (1995).
45. Ho, H. O., Hsiao, C. C. & Sheu, M. T. Preparation of microemulsions using polyglycerol fatty acid esters as surfactant for the delivery of protein drugs. Journal of Pharmaceutical Sciences 85, 138-143 (1996).
46. Silva-Cunha, A., Grossiord, J. L., Puisieux, F. & Seiller, M. W/O/W multiple emulsions of insulin containing a protease inhibitor and an absorption enhancer: preparation, characterization and determination of stability towards proteases in vitro. International Journal of Pharmaceutics 158, 79-89 (1997).
47. Cournarie, F. et al. Improved formulation of W/O/W multiple emulsion for insulin encapsulation. Influence of the chemical structure of insulin. Colloid Polymer Science 282, 562-568 (2004).
48. Morishita, I., Morishita, M., Takayama, K., Machida, Y. & Nagai, T. Enteral insulin delivery by microspheres in 3 different formulations using Eudragit L-100 and S-100. International Journal of Pharmaceutics 91, 29-37 (1993).
49. Trenktrog, T., Muller, B. W., Specht, F. M. & Seifert, J. Enteric coated insulin pellets: development, drug release and in vivo evaluation. European Journal Pharmaceutical Sciences 4, 323-329 (1996).
50. Hosny, E. A., Ghilzai, N. M. K. & Al-Dhawalie, A. H. Effective intestinal absorption of insulin in diabetic rats using enteric coated capsules containing sodium salicylate. Drug Development and Industrial Pharmacy 21, 1583-1589 (1995).
51. Saffran, M. et al. A new approach to the oral administration of insulin and other peptide drugs. Science Reports 233, 1081-1084 (1986).
52. Tozaki, H. et al. Degradation of insulin and calcitonin and their protection by various protease inhibitors in rat caecal contents: implications in peptide delivery to the colon. Journal of Pharmacy and Pharmacology 49, 164-168 (1997).
53. Saffran, M., Pansky, B., Budd, G. C. & Williams, F. E. Insulin and the gastrointestinal tract. Journal of Controlled Release 46, 89-98 (1997).
54. McPhillips, A., Uraizee, S., Ritschel, W. & Sakr, A. Evaluation of fluid-bed applied acrylic polymers for the targeted peroral delivery of insulin. S.T.P. Pharma. Sciences 7, 476-482 (1997).
55. Carino, G. P., Jacob, J. S. & Mathiowitz, E. Nanosphere based oral insulin delivery. Journal of Controlled Release 65, 261-269 (2000).
56. Kimura, T. et al. Oral administration of insulin as poly(vynil alcohol).gel spheres in diabetic rats. Biology Pharmaceutical Bulletin 19, 897-900 (1996).
57. Manosroi, A. & Manosroi, J. Microencapsulation of human insulin DEAE-dextran complex and the complex in liposomes by the emulsion non-solvent addition method. Journal of Microencapsulation 14, 761-768 (1997).
58. Morishita, M. et al. The dose-related hypoglycemic effects of insulin emulsions incorporating highly purified EPA and DHA. International Journal of Pharmaceutics 201, 175-185 (2000).
59. Senthil Rajan D, M. U., Veeran Gowda K, Bose A, Ganesan M, Pal T K. Oral delivery system of insulin microspheres: effect on relative hypoglycemia of diabetic albino rats. Bollettino Chimico Farmaceutico 143, 315-318. (2004).
60. Mesiha, M. & Sidhom, M. Increased oral absorption enhancement of insulin by medium viscosity hydroxypropyl cellulose. International Journal of Pharmaceutics 114, 137-140 (1995).
61. Ziv, E. et al. Oral administration of insulin in solid form to nondiabetic and diabetic dogs. Journal of Pharmaceutical Sciences 83, 792-794 (1994).
62. Peppas, N. A. & Kavimandan, N. J. Nanoscale analysis of protein and peptide absorption: Insulin absorption using complexation and pH-sensitive hydrogels as delivery vehicles. European Journal of Pharmaceutical Sciences Advances in Understanding Oral Absorption and Delivery of Problem Compounds—Selected Papers from the 3rd World Conference on Drug Absorption, Transport and Delivery 29, 183-197 (2006).
63. Greenley, R. Z., Brown, T. M., Vogt, C. E., Zia, H., Rodgers, R. L., Christie M. & Luzzi, L. A. Polymer matrices for oral delivery. Polymer Preprints 31, 182 (1990).
64. Damge, C., Michel, C., Aprahamian, M., Couvreur, P. & Devissaguet, J.-P. Nanocapsules as carriers for oral peptide delivery. Journal of Controlled Release 13, 233-239 (1990).
65. Reis, C. P., Neufeld, R. J., Vilela, S., Ribeiro, A. J. & Veiga, F. Review and current status of emulsion/dispersion technology using an internal gelation process for the design of alginate particles. Journal of Microencapsulation 23, 245-257 (2006).
66. Lencki, R. W. J., Neufeld, R. J. & Spinney, T. in U.S. Pat. No. 48,225,341,989.
67. Couvreur, P., Dubernet, C. & Puisieux, F. Controlled drug delivery with nanoparticles: current possibilities and future trends. European Journal of Pharmaceutics and Biopharmaceutics 41, 2-13 (1995).
68. Couvreur, P. Polyalkylcyanoacrylates as colloidal drug carriers. CRC Critical Reviews Therapeutical Drug Carrier System 5, 1-20 (1988).
69. Allémann, E., Gurny, R. & Doekler, E. Drug-loaded Nanoparticles-Preparation methods and drug targeting issues. European Journal of Pharmaceutics and Biopharmaceutics 39, 173-191 (1993).
70. Brannon-Peppas, L. & Blanchette, J. O. Nanoparticle and targeted systems for cancer therapy. Advanced Drug Delivery Reviews 56, 1649-1659 (2004).
71. Kreuter, J., Stieneker, F. & Lower, J. Poly(methyl methacrylate) nanoparticles as effective adjuvants for HIV-antigens. Proc. Int. Symp. Contr. Rel. Bioact. Matter. 18, 277-278 (1991).
72. Lemoine, D. & Preat, V. Polymeric nanoparticles as delivery system for influenza virus glycoproteins. Journal of Controlled Release 54, 15-27 (1998).
73. Aynié, I., Vauthier, C., Fattal, E., Foulquier, M. & Couvreur, P. in Future strategies for drug delivery with pariculate systems. (ed. R. H. Muller et al.) 11-16 (CRC Press, Boca Raton, 1998).
74. Mao, H.-Q. et al. Chitosan-DNA nanoparticles as gene carriers:synthesis, characterization and transfection efficiency. Journal of Controlled Release 70, 399-421 (2001).
75. Prabha, S., Zhou, W.-Z., Panyam, J. & Labhasetwar, V. Size-dependency of nanoparticle-mediated gene transfection studies with fractionated nanoparticles. International Journal of Pharmaceutics 244, 105-115 (2002).
76. Panyam, J. & Labhasetwar, V. Biodegradable nanoparticles for drug and gene delivery to cells and tissue. Advanced Drug Delivery Reviews 55, 329-347 (2003).
77. Reis, C. P., Neufeld, R. J., Ribeiro, Antonio J. & Veiga, F. Nanoencapsulation I. Methods for preparation of drug-loaded polymeric nanoparticles. Nanomedicine: Nanotechnology, Biology and Medicine 2, 8-21 (2006).
78. Norris, D. A., Puri, N. & Sinko, P. J. Effect of physical barriers and properties on the oral absorption of particulates. Advanced Drug Delivery Reviews 34, 135-154 (1998).
79. Morishita, M. et al. Novel oral insulin delivery systems based on complexation polymer hydrogels: Single and multiple administration studies in type 1 and 2 diabetic rats. Journal of Controlled Release 110, 587-594 (2006).
80. Shechter, Y. et al. Albumin-Insulin Conjugate Releasing Insulin Slowly under Physiological Conditions: A New Concept for Long-Acting Insulin. Bioconjugate Chemistry 16, 913-920 (2005).
81. Hovgaard, L., Jacobs, H., Wilson, D. E. & Kim, S. W. Stabilization of insulin by alkylmaltosides. B. Oral absorption in vivo in rats. International Journal of Pharmaceutics 132, 115-121 (1996).

1. An oral submicron particle delivery system for proteins which comprises:
a. a core comprising said protein to be immobilized, a naturally occuring immobilizing agent, an adjuvant, and an immobilizing agent crosslinker to obtain gelled submicron particles by an emulsification-based method;
b. a primary coating material surrounding said core which comprises a blend of hydrophilic, natural and biodegradable polymers;
c. a secondary coating material surrounding said primary coating wherein said secondary coating material comprises protein material.

2. The system according to claim 1, wherein said protein comprises unmodified human insulin as a drug to be immobilized in said immobilizing agent.

3. The system according to claim 1, wherein said immobilizing agent comprises a naturally-occuring polysaccharide.

4. The system according to claim 1, wherein said immobilizing agent comprises a sodium alginate which gels in presence of divalent ions.

5. The system, according to claim 1, wherein said immobilizing agent comprises a polyanionic polymer at pH 4.5.

6. The system according to claim 1, wherein immobilizing agent crosslinker is calcium released from calcium complex.

7. The system according to claim 1, wherein the immobilizing agent crosslinker comprises calcium carbonate.

8. An emulsification-based process for production of an oral submicron particle delivery system for proteins, of claim 1, which comprises:
a. providing an aqueous phase, internal phase, containing an immobilizing agent, an adjuvant, immoblized protein and an immobilizing agent crosslinker to cause gelation of said immobilizing agent,
b. contacting said internal phase with a hydrophobic liquid, external phase, under mild conditions leading to formation of droplets of said internal phase in said external phase, and
c. adding an oil-soluble organic acid to mixture obtained in step b) to convert said droplets into gel particles,
d. recovering the resultant gelled submicron particles,
e. primary coating the recovered gelled submicron particles using a blend of hydrophilic polymers with high calcium levels, and
f. coating the primary coated submicron particles in step e) using protein coating material.

9. The process according to claim 8, which comprises introducing protein to be immobilized into said immobilizant agent and the adjuvant so as to obtain solid containing said protein, said immobilizing agent and said adjuvant.

10. The process according to claim 1, wherein immobilizing agent crosslinker is calcium released from calcium complex.

11. The process according to claim 10, which comprises adding a pH-decreasing compound, which dissolve the calcium complex, to said mixture b).

12. The process according to claim 8, wherein said adjuvant comprises dextran sulfate.

13. The process according to claim 8, wherein said hydrophobic liquid is paraffin oil.

14. The process according to claim 8, wherein said oil-soluble acid is acetic acid.

15. The process according to claim 8, further comprising removing residual oil using partition phases, centrifuge and thereby to produce a colloidal suspension of particles.

16. The system according to claim 8, wherein said blend of hydrophilic polymers comprises chitosan acetate at a concentration of about 0.015% to 0.15% (w/w), polyethyleneglycol at a concentration of about 0.0375% to 0.3% (w/w) and calcium chloride at a concentration of about 1.5% (w/w) at pH 4.5.

17. The process according to claim 8, wherein said protein coating material is albumin at a concentration of about 0.5% to 1.5% (w/w) at pH 5.1.
